(19) United States
(12) Patent Application Publication
(10) Pub. No.: US 2008/0241090 A1
(43) Pub. Date: Oct. 2, 2008

(54) Use of Fluorescent Perylene Compounds for the Treatment of Human Hair

(75) Inventors: Markus Speckbacher, Aschaffenburg (DE); Jan Baumeister, Farvagny-le-Grand (CH)

Correspondence Address: MICHAEL J. STRIKER, 103 EAST NECK ROAD, HUNTINGTON, NY, 11743, US

(73) Assignee: WELLA AKTIENGESELLSCHAFT, Darmstadt (DE)

(21) Appl. No.: 11/570,627

(22) PCT Filed: Jun. 1, 2005
(86) PCT No.: PCT/EP05/05860

§ 371 Date: Dec. 14, 2006

(30) Foreign Application Priority Data
Jun. 17, 2004 (DE) 10 2004 029 385.6

(51) Int. Cl. A61K 8/49; C07D 221/02; A61Q 5/00

(52) U.S. Cl. 424/70.1; 546/34

(57) ABSTRACT

The invention relates to the use of fluorescent perylene compounds for the treatment of human hair, in particular for improving the shine of hair, and to corresponding hair-treatment compositions. Preferred perylene compounds are N-alkyl-substituted 3,4:9,10-bis(dicarboximide)perylenes.

The invention relates to the use of fluorescent perylene compounds for the treatment of human hair, in particular for improving the shine of hair, and to corresponding hair-treatment compositions.

In the fast-moving hairstyling market, the need for products with new properties is very great. Product innovations which appeal to several senses at the same time, awaken positive associations or differ from conventional products by virtue of extraordinary additional properties have a high potential for attracting attention. Of interest are, for example, hairstyling products which, besides the primarily desired styling effect, additionally exhibit unusual color effects, e.g. fluorescent effects on human hair, or improve the shine of the hair or the luminosity of the hair color. However, many known fluorescent dyes are unsatisfactory particularly in hydrophobic styling products such as, for example, hair waxes, since, for example, the effect is inadequate, or the cosmetic compatibility or the compatibility with product ingredients is not completely satisfactory.

Furthermore, in hair cosmetics, the hair shine of human hair and the luminosity of hair colors are of great importance. Shiny hair is usually associated with healthy, natural, vital hair. There are a number of products for improving hair shine. Although many products, e.g. based on oils or silicone compounds, improve hair shine, they have disadvantageous side-effects at the same time. In particular, they can weigh the hair down, which hinders applicability on fine hair. There is therefore a need for further products which improve hair shine, color effects on the hair or the luminosity of hair colors.

It has now been found that fluorescent perylene compounds are very well suited to treating human hair, in particular for producing shine effects and fluorescent color effects. The invention therefore provides the use of fluorescent perylene compounds for the cosmetic treatment of human hair, in particular the use for improving the shine or the luminosity of human hair.

Fluorescent perylene compounds are substances which have at least one perylene structural unit and are able to absorb light of a wavelength which is preferably in the UV region (<400 nm) and radiate light of a greater wavelength which is preferably in the visible region (>400 nm).

Preferred perylene compounds are those which are substituted in at least one, preferably two, of the positions 3, 4, 9 or 10 by a carbonyl group. Carbonyl groups are, for example, keto groups, carboxylic acid groups, carboxylic esters, carboxamides or carboximides. Preferably, the perylene compound is substituted by a group bridging positions 3, 4 and/or positions 9, 10.

The bridging group here may be a group —CO—NR1-CO—, where R1 is linear or branched, unsubstituted or substituted hydrocarbon group, in particular alkyl, cycloalkyl, aryl, alkylaryl or arylalkyl groups having 1 to 24 carbon atoms, or R1 is chosen from

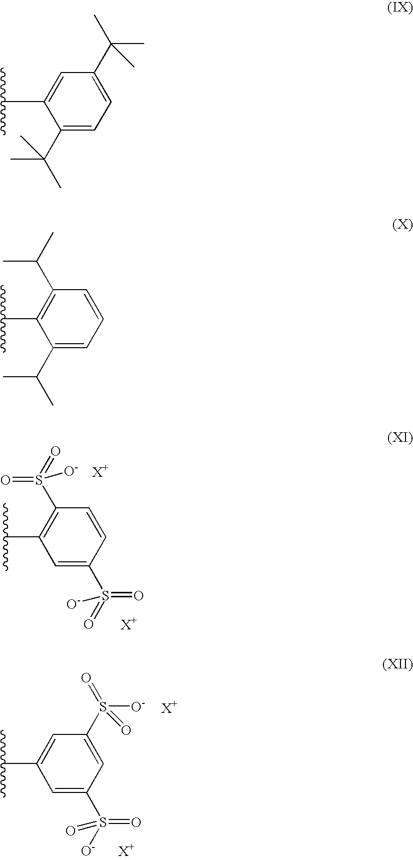

where X⁺ is a proton or a cation, where lithium ions, sodium ions, potassium ions, ammonium ions, magnesium ions or calcium ions are preferred;

the bridging group can, for example, also be chosen from

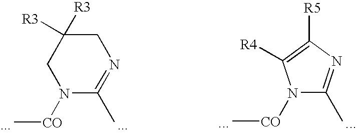

where R3 is hydrocarbon groups, in particular linear C1-C9-alkyl groups; R4 and R5 together form a fused-on hydrocarbon radical. The fused-on radical can, for example, be chosen from the formulae

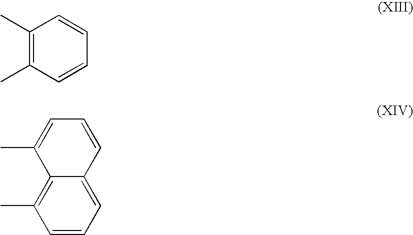

Further perylene compounds are those of the formulae (I) to (VIII) and (XIX).

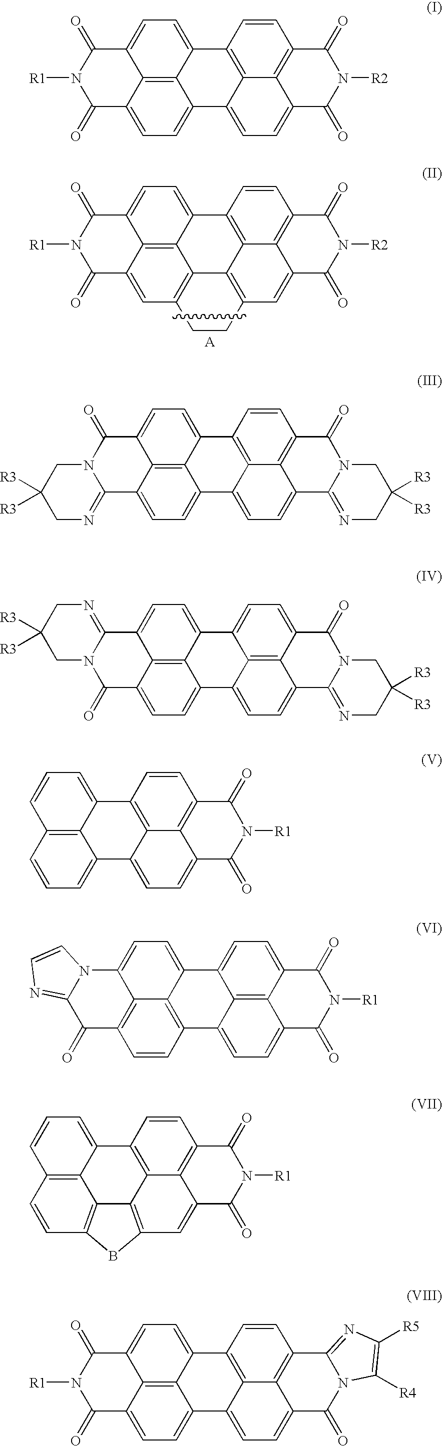

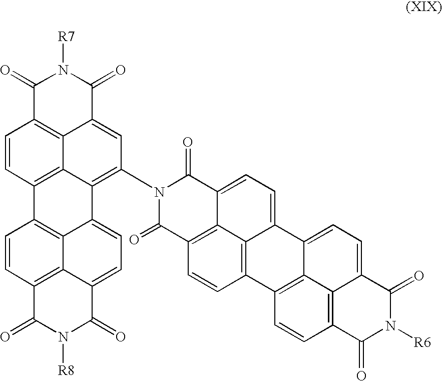

in which R1 and R2, independently of one another, are linear or branched, unsubstituted or substituted hydrocarbon groups, in particular alkyl, cycloalkyl, aryl, alkylaryl or arylalkyl groups having 1 to 24 carbon atoms or are chosen from radicals of the formulae (IX) to (XII) according to the abovementioned definition and R3, R4 and R5 have the same meaning as above and in which R6, R7 and R8, independently of one another, are linear or branched C₁-C₂₄-alkyl groups.

A is a divalent connecting group, e.g. a sulfur atom, an NH group or a compound chosen from the group consisting of the organic radicals of the formulae (XV) to (XVIII),

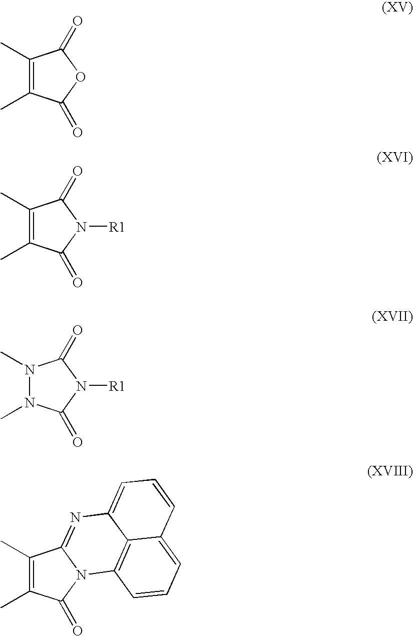

in which R₁ has the abovementioned meaning.

B is a divalent connecting group, e.g. a sulfur atom or an H group.

Suitable fluorescent perylene derivatives of the general formulae (I) to (VIII) which may be mentioned are, for example:

N,N′-bis(alkyl)perylene-3,4:9,10-bis(dicarboximide), e.g. N,N′-bis(1-butylpentyl)perylene-3,4:9,10-bis(dicarboximide), N,N′-bis(1-hexylheptyl)perylene-3,4:9,10-bis(dicarboximide), N,N′-bis(1-heptyloctyl)-perylene-3,4:9,10-bis(dicarboximide), N,N′-bis(1-octylnonyl)perylene-3,4:9,10-bis (dicarboximide), or N,N′-bis(2,5-di-tert-butylphenyl)perylene-3,4:9,10-bis(dicarboximide); N-alkylperylene-3,4-dicarboximide, e.g. N-(1-hexyl-heptyl)perylene-3,4-dicarboximide or N-(2,5-di-tert-butylphenyl)perylene-3 4-dicarboximide; N,N′-bis(alkyl)benzo[ghi]perylene-2,3,8,9,11,12-hexacarboxylic 2,3;8,9-bis(dicarboximide)11,12-anhydride, e.g. N,N′-bis(1-hexylheptyl)benzo[ghi]-perylene-2,3,8,9,11,12-hexacarboxylic 2,3;8,9-bis-(dicarboximide) 11,12-anhydride; N,N′-bis(alkyl)benzo[ghi]perylene-2,3,8,9,11,12-hexa-carboxylic tris(dicarboximide), e.g. N,N′-bis(1-hexylheptyl)benzo[ghi]perylene-2,3,8,9,11,12-hexacarboxylic tris(dicarboximide); N,N′-bis(alkyl)thieno[2′,3′,4′,5′:4,5]phenanthro-[2,1,10-def:7,8,9-d′e′f′]diisoquinolino-2H,7H-1,3,6,8-tetrone, e.g. N,N′-bis(2,5-di-tert-butylphenyl)thieno-[2′,3′,4′,5′:4,5]phenanthro[2,1,10-def:7,8,9-d′e′f′]-diisoquinolino-2H,7H-1,3,6,8-tetrone and N,N′-bis(hexylheptyl)thieno[2′,3′,4′,5′:4,5]phenanthro-[2,1,10-def:7,8,9-d′e′f′]diisoquininolino-2H,7H-1,3,6,8-tetrone; where the alkyl groups are in each case linear or branched, unsubstituted or substituted alkyl, cycloalkyl, aryl, alkylaryl or arylalkyl groups having 1 to 24 carbon atoms.

A suitable fluorescent perylene derivative of the formula (XIX) is, for example, 1-[N^{1′}-(N^{2′}-(alkyl)-perylene-3,4:9,10-bis(dicarboximide)]-N¹,N²-bis(alkyl)-perylene-3,4:9,10-bis(dicarboximide), where the alkyl groups may be linear or branched, unsubstituted or substituted alkyl, cycloalkyl, aryl, alkylaryl or arylalkyl groups having 1 to 24 carbon atoms.

Fluorescent perylene compounds and their preparation are known per se and are described, for example, in DE 37 034 95; DE 196 51 712; WO 00/23446; H. Langhals, S. Kirner, “Novel Fluorescent Dyes by the Extension of the Core of Perylene-tetracarboxylic Bisimides”, Eur. J. Org. Chem. 2000, 365-380; H. Langhals, H. Jaschke, U. Ring, P. von Unold, “Imidazolo Perylene Imides: a Highly Fluorescent and Stable Replacement of Terrylene”, Angew. Chem. 1999, 111, 143-145; Angew. Chem. Int. Ed. Engl. 1999, 38, 201-203; H. Langhals, H. Bastani-Oskoui, “Synthesis of Readily Soluble Tetraazaviolanthrone and -isoviolanthrone Fluorescent Dyes”, J. Prakt. Chem, 1997, 339, 597-602; L. Feiler, H. Langhals, K. Polborn, “Synthesis of Perylene-3,4-dicarboximides—Novel, Highly Photostable Fluorescent Dyes”, Liebigs Ann. Chem. 1995, 1229-1244; H. Langhals, S. Sprenger, M.-T. Brandherm, “Peryleneamidine-imide Dyes”, Liebigs Ann. Chem. 1995, 481-486; S. Demmig, H. Langhals, “Leichtlösliche, lichtechte Perylen-Fluoreszenzfarbstoffe” [Readily soluble, lightfast perylene fluorescent dyes], Chem. Ber. 1988, 121, 2257 230. In this regard, reference is made to these documents.

The invention also provides the hitherto unknown perylene compounds of the abovementioned formula (XIX) and in particular 1-[N¹—(N^{2′}-(alkyl)perylene-3,4:9,10-bis(dicarboximide)]-N¹,N²-bis(alkyl)perylene-3,4:9,10-bis(dicarboximide). These can be prepared, for example, by condensation reaction from 1-amino-N,N′-bis(alkyl)-perylene-3,4:9,10-bis(dicarboximide) and N-(alkyl)-perylene-3,4:9,10-tetracarboxylic 3,4-anhydride 9,10-carboximide, which are known per se, optionally in the presence of suitable catalysts. An exemplary reaction scheme with a 1-hexylheptyl group as alkyl group is as follows:

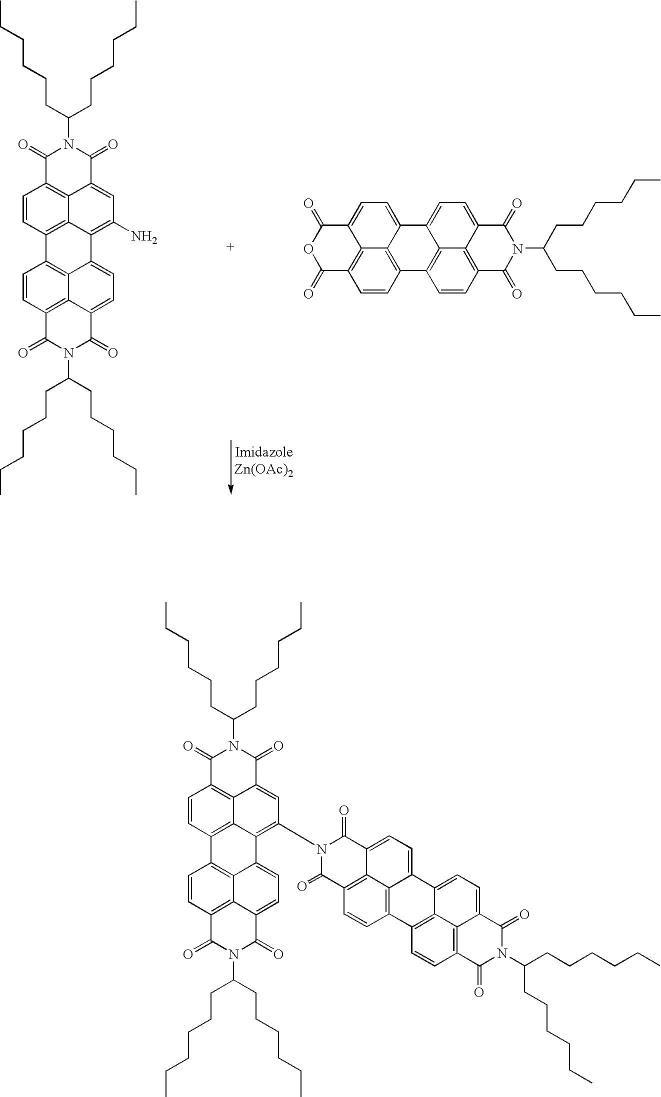

According to the invention, the fluorescent perylene compounds can be used for producing compositions for the treatment of human hair. The hair-treatment compositions according to the invention may be compositions for cleaning hair, for the care of hair or for the temporary shaping and/or stabilization of the hairstyle (styling compositions), which can be applied in highly diverse types of application, e.g. as leave-on or as rinse-off products. Application forms are, for example, shampoos, hair treatments, hair rinses, hair lotions, end fluids, hair oils, brilliantines, hair sprays, hair lacquers, setting lotions, hair foams, hair gels, hair waxes, hair creams etc.

Hair cosmetic compositions according to the invention comprise the fluorescent perylene compounds preferably in an amount of from 0.01 to 20% by weight, in particular from 0.1 to 15% by weight or from 0.3 to 10% by weight. Further active ingredients and additives are present, depending on the type and intended use, preferably in an amount of from 0.01 to 20% by weight, in particular from 0.05 to 10% by weight or from 0.1 to 5% by weight.

The compositions according to the invention can be used to achieve a particular luminosity of the hair treated therewith, particularly when using fluorescent perylene compounds with high quantum yields (e.g. 50-100%). In one embodiment, the composition according to the invention is present in the form of a hair wax, i.e. it has a wax-like consistency and comprises at least one wax substance in an amount of from preferably 0.5 to 30% by weight, and optionally further water-insoluble substances. The wax-like consistency is preferably characterized in that the needle penetration number (measurement unit 0.1 mm, test weight 100 g, test time 5 s, test temperature 25° C.; in accordance with DIN 51 579) is greater than or equal to 10, particularly preferably greater than or equal to 20, and that the solidification point of the product is preferably greater than or equal to 30° C. and less than or equal to 70°0 C., particularly preferably in the range from 40 to 55° C.

In principle, the wax substance which can be used is any wax known in the prior art. These include animal, vegetable, mineral and synthetic waxes, microcrystalline waxes, macrocrystalline waxes, solid paraffins, petrolatum, vaseline, ozokerite, montan wax, Fischer-Tropsch waxes, polyolefin waxes, e.g. polybutene, beeswax, wool wax and derivatives thereof, such as, for example, wool wax alcohols, candelilla wax, olive wax, carnauba wax, Japan wax, apple wax, hydrogenated fats, fatty acid esters, fatty acid glycerides, wax-like emulsifiers with a HLB value below 7, fatty alcohols, fatty acids or hydrophilic waxes, such as, for example, high molecular weight polyethylene glycols with a molecular weight of from 800 to 20 000, preferably from 2000 to 10 000 g/mol, polyethylene waxes and silicone waxes. The wax substances have a solidification point of preferably above 40° C,, in particular above 55° C. The needle penetration number (0.1 mm, 100 g, 5 s, 25° C.; in accordance with DIN 51 579,) is preferably in the range from 2 to 70, in particular from 3 to 40.

In addition to the wax substances, liquid, hydrophobic oils may be present. The oils preferably have a melting point of less than or equal to 25° C. and a boiling point of preferably above 250° C., in particular above 300° C. In principle, any oil generally known to the person skilled in the art can be used here. Of suitability are vegetable or animal oils, mineral oils (Paraffinum liquidum), silicone oils or mixtures thereof. Hydrocarbon oils, e.g. paraffin or isoparaffin oils, squalane, oils of fatty acids and polyols, in particular triglycerides, are suitable. Suitable vegetable oils are, for example, sunflower oil, coconut oil, castor oil, lanolin oil, jojoba oil, corn oil, soybean oil.

In one embodiment, the composition according to the invention is present as a haircare composition, e.g. as a hair treatment or as a hair lotion and additionally comprises at least one haircare substance. The haircare substance is preferably present in an amount of from 0.01 to 20% by weight, particularly preferably from 0.05 to 10% by weight, very particularly preferably from 0.1 to 5% by weight. Haircare substances are, for example, chosen from cationic surfactants; cationic polymers; betaine; panthenol; panthenyl ethyl ether; sorbitol; protein hydrolyzates; plant extracts or one of the abovementioned oils or wax substances.

Suitable cationic surfactants contain amino groups or quaternized hydrophilic ammonium groups which, in solution, carry a positive charge and can be represented by the general formula

N⁽⁺⁾R¹R²R³R⁴ X⁽⁻⁾

where R1 to R4, independently of one another, are aliphatic groups, aromatic groups, alkoxy groups, polyoxyalkylene groups, alkylamido groups, hydroxyalkyl groups, aryl groups or alkaryl groups having 1 to 22 carbon atoms, where at least one radical has at least 6, preferably at least 8, carbon atoms, and X⁻ is an anion, for example a halogen, acetate, phosphate, nitrate or alkyl sulfate, preferably a chloride. In addition to the carbon atoms and the hydrogen atoms, the aliphatic groups can also contain crosslinkages or other groups, such as, for example, further amino groups. Examples of suitable cationic surfactants are the chlorides or bromides of alkyldimethylbenzylammonium salts, alkyltrimethylammonium salts, e.g. cetyltrimethylammonium chloride or bromide, tetradecyltrimethylammonium chloride or bromide, alkyldimethylhydroxyethylammonium chlorides or bromides, the dialkyldimethylammonium chlorides or bromides, alkylpyridinium salts, for example lauryl- or cetylpyridinium chloride, alkylamidoethyltrimethylammonium ether sulfates, and compounds with cationic character, such as amine oxides, for example alkylmethylamine oxides or alkylaminoethyldimethylamine oxides. Particular preference is given to C8-22-alkyldimethylbenzylammonium compounds, C8-22 -alkyltrimethylammonium compounds, in particular cetyltrimethylammonium chloride, C8-22-alkyldimethylhydroxyethylammonium compounds, di(C8-22-alkyl)dimethylammonium compounds, C8-22-alkylpyridinium salts, C8-22-alkylamidoethyltrimethylammonium ether sulfates, C8-22-alkylmethylamine oxides, C8-22-alkyl-aminoethyldimethylamine oxides.

Besides the abovementioned cationic surfactants, further suitable cationic or amine-substituted surfactants are those of the formula R1-NH—(CH₂)n-NR2R3 or of the formula R1-NH—(CH₂)n-N⁺R2R3R4 X⁻ in which R1 is an acyl radical or an alkyl radical having 8 to 24 carbon atoms, which may be branched or unbranched, saturated or unsaturated, where the acyl radical and/or the alkyl radical can contain one or more OH groups, R2, R3 and R4, independently of one another, are hydrogen, alkyl or alkoxyalkyl radicals having 1 to 6 carbon atoms, which may be identical or different, saturated or unsaturated and substituted by one or more hydroxy groups, X⁻ is an anion, in particular a halide ion or a compound of the general formula RSO₃⁻, in which R has the meaning of saturated or unsaturated alkyl radicals having 1 to 4 carbon atoms, and n is an integer between 1 and 10, preferably from 2 to 5.

Preferably, the haircare active ingredient is an amidoamine and/or a quaternized amidoamine of the abovementioned formulae, in which R1 is a branched or unbranched, saturated or unsaturated acyl radical having 8 to 24 carbon atoms, which can comprise at least one OH group. Preference is also given to those amines and/or quaternized amines in which at least one of the radicals R2, R3 and R4 is a radical according to the general formula CH₂CH₂OR5, in which R5 can have the meaning of alkyl radicals having 1 to 4 carbon atoms, hydroxyethyl or H. Suitable amines or amidoamines, which may optionally be quaternized, are, in particular, those with the INCI names Ricinoleamidopropyl Betaine, Ricinoleamidopropyl Dimethylamine, Ricinoleamidopropyl Dimethyl Lactate, Ricinoleamidopropyl Ethyldimonium Ethosulfate, Ricinoleamidopropyltrimonium Chloride, IRicinoleamidopropyltrimonium Methosulate, Cocamidopropyl Betaine, Cocamidopropyl Dimethylamine, Cocamidopropyl Ethyldimonium Ethosulfate, Cocamidopropyltrimonium Chloride, Behenamidopropyl Dimethylamine, Isostearylamidopropyl Dimethylamine, Stearylamidopropyl Dimethylamine, Quaternium-33, Undecyleneamidopropyltrimonium Methosulfate.

In one embodiment, the composition according to the invention comprises, as haircare or hair-setting additive, at least one cationic polymer, i.e. a polymer with cationic or cationizable groups, in particular primary, secondary, tertiary or quaternary amine groups in an amount of from preferably 0.01 to 20% by weight or from 0.05 to 10% by weight, particularly preferably from 0.1 to 5% by weight. The cationic charge density is preferably 1 to 7 meq/g.

The suitable cation-active polymers are preferably hair-setting or hair-conditioning polymers. Suitable polymers preferably comprise quaternary amine groups. The cationic polymers may be homopolymers or copolymers, where the quaternary nitrogen groups are present either in the polymer chain or preferably as substituent on one or more of the monomers. The monomers containing ammonium groups may be copolymerized with noncationic monomers. Suitable cationic monomers are unsaturated, free-radically polymerizable compounds which carry at least one cationic group, in particular ammonium-substituted vinyl monomers, such as, for example, trialkylmethacryloxyalkylammonium, trialkylacryloxyalkylammonium, dialkyldiallylammonium and quaternary vinylammonium monomers with cyclic, cationic nitrogen-containing groups, such as pyridinium, imidazolium or quaternary pyrrolidones, e.g. alkylvinylimidazolium, alkylvinylpyridinium, or alkylvinylpyrrolidone salts. The alkyl groups of these monomers are preferably lower alkyl groups, such as, for example, C1- to C7-alkyl groups, particularly preferably C1- to C3-alkyl groups.

The monomers containing ammonium groups may be copolymerized with noncationic monomers. Suitable comonomers are, for example, acrylamide, methacrylamide, alkyl- and dialkylacrylamide, alkyl- and dialkylmethacrylamide, alkyl acrylate, alkyl methacrylate, vinylcaprolactone, vinylcaprolactam, vinylpyrrolidone, vinyl esters, e.g. vinyl acetate, vinyl alcohol, propylene glycol or ethylene glycol, where the alkyl groups of these monomers are preferably C1- to C7-alkyl groups, particularly preferably C1- to C3-alkyl groups.

Suitable polymers with quaternary amine groups are, for example, the polymers described in the CTFA Cosmetic Ingredient Dictionary under the names Polyquaternium, such as methylvinylimidazolium chloride/vinylpyrrolidone copolymer (Polyquaternium-16) or quaternized vinylpyrrolidone/dimethylaminoethyl methacrylate copolymer (Polyquaternium-11), and quaternary silicone polymers and oligomers, such as, for example, silicone polymers with quaternary end groups (Quaternium-80).

Preferred Synthetic-Based Cationic Polymers:

Poly(dimethyldiallylammonium chloride); copolymers of acrylamide and dimethyldiallylammonium chloride; quaternary ammonium polymers formed by the reaction of diethyl sulfate and a copolymer of vinylpyrrolidone and dimethylaminoethyl methacrylate, in particular vinylpyrrolidone/dimethylaminoethyl methacrylate methosulfate copolymer (e.g. Gafquat® 755 N. Gafquat® 734); quaternary ammonium polymers of methylvinylimidazolium chloride and vinylpyrrolidone (e.g. LUVIQUAT® HM 550); Polyquaternium-35; Polyquaternium-57; polymer of trimethylammonium ethyl methacrylate chloride; terpolymers of dimethyldiallylammonium chloride, sodium acrylate and acrylamide (e.g. Merquat® Plus 3300); copolymers of vinylpyrrolidone, dimethylaminopropyl methacrylamide and methacryloylaminopropyllauryldimethylammonium chloride; terpolymers of vinylpyrrolidone, dimethylaminoethyl methacrylate and vinylcaprolactam (e.g. Gaffix® VC 713); vinylpyrrolidone/methacrylamidopropyltrimethylammonium, chloride copolymers (e.g. Gafquat® HS 100); copolymers of vinylpyrrolidone and dimethylaminoethyl methacrylate; copolymers of vinylpyrrolidone, vinylcaprolactam and dimethylaminopropylacrylamide; polyesters or oligoesters constructed from at least one first monomer type which is chosen from hydroxy acid substituted by at least one quaternary ammonium group; dimethylpolysiloxanes terminally substituted by quaternary ammonium groups.

Suitable cationic polymers which are derived from natural polymers are, in particular, cationic derivatives of polysaccharides, for example cationic derivatives of cellulose, starch or guar. Also suitable are chitosan and chitosan derivatives. Cationic polysaccharides have, for example, the general formula

G-O—B—N⁺R^{a}R^{b}R^{c} X⁻

G is an anhydroglucose radical, for example starch or cellulose anhydroglucose;

B is a divalent connecting group, for example alkylene, oxyalkylene, polyoxyalkylene or hydroxyalkylene;

R^{a}, R^{b} and R^{c}, independently of one another, are alkyl, aryl, alkylaryl, arylalkyl, alkoxyalkyl or alkoxyaryl in each case having up to 18 carbon atoms, where the total number of the carbon atoms in R^{a}, R^{b} and R^{c} is preferably at most 20;

X is a customary counteranion, for example a halogen, acetate, phosphate, nitrate or alkyl sulfate, preferably a chloride. Cationic celluloses are, for example, those with the INCI names Polyquaternium-10 or Polyquaternium-24. A suitable cationic guar derivative has, for example, the INCI name Guar Hydroxypropyltrimonium Chloride.

Particularly preferred cation-active substances are chitosan, chitosan salts and chitosan derivatives. The chitosans to be used according to the invention are completely or partially deacetylated chitins. The molecular weight can be spread over a broad spectrum, for example from 20 000 to about 5 million g/mol, e.g. from 30 000 to 70 000 g/mol. Preferably, however, the molecular weight is more than 100 000 g/mol, particularly preferably from 200 000 to 700 000 g/mol. The degree of deacetylation is preferably 10 to 99%, particularly preferably 60 to 99%. A preferred chitosan salt is chitosonium pyrrolidonecarboxylate, e.g. Kytamer® PC with a molecular weight of from about 200 000 to 300 000 g/mol and deacetylation of from 70 to 85%. Suitable chitosan derivatives are quaternized, alkylated or hydroxyalkylated derivatives, e.g. hydroxyethyl-, hydroxypropyl- or hydroxybutylchitosan. The chitosans or chitosan derivatives are preferably in neutralized or partially neutralized form. The degree of neutralization is preferably at least 50%, particularly preferably between 70 and 100%, based on the number of free base groups. Neutralizing agents which can be used are, in principle, all cosmetically compatible inorganic or organic acids, such as, for example, formic acid, tartaric acid, malic acid, lactic acid, citric acid, pyrrolidonecarboxylic acid, hydrochloric acid etc., of which pyrrolidonecarboxylic acid is particularly preferred.

Preferred Natural-Based Cationic Polymers:

Cationic cellulose derivatives of hydroxyethylcellulose and diallyldimethylammonium chloride; cationic cellulose derivatives of hydroxyethylcellulose and epoxide substituted by trimethylammonium; chitosan and salts thereof; hydroxyalkylchitosans and salts thereof; alkylhydroxyalkylchitosans and salts thereof; N-hydroxyalkylchitosan alkyl ethers.

In one embodiment, the composition according to the invention is present in the form of a gel, a viscous lotion or in the form of a spray gel which is sprayed using a mechanical device and comprises at least one gel former, preferably at least one gel-forming, thickening polymer. The gel former is present in an amount of from preferably 0.05 to 10% by weight, particularly preferably from 0.1 to 5% by weight. The gel-like or viscous product has a viscosity of preferably at least 250 mPa s (measured using a Bohlin Rheometer CS, measurement body C25 at 25° C. and a shear rate of 50 s⁻¹). The viscosity of the gel is preferably from 500 to 50 000 mPa s, in particular from 1000 to 15 000 mPa s at 25° C. Gel formers are, for example, chosen from copolymers of at least one first type of monomer, which is chosen from acrylic acid and methacrylic acid, and at least one second type of monomer, which is chosen from esters of acrylic acid and ethoxylated fatty alcohol; crosslinked polyacrylic acid; crosslinked copolymers of at least one first type of monomer, which is chosen from acrylic acid and methacrylic acid, and at least one second type of monomer, which is chosen from esters of acrylic acid with C10- to C30-alcohols; copolymers of at least one first type of monomer, which is chosen from acrylic acid and methacrylic acid, and at least one second type of monomer, which is chosen from esters of itaconic acid and ethoxylated fatty alcohol; copolymers of at least one first type of monomer, which is chosen from acrylic acid and methacrylic acid, at least one second type of monomer, which is chosen from esters of itaconic acid and ethoxylated C10- to C30-alcohol, and a third type of monomer, chosen from C1- to C4-aminoalkyl acrylates; copolymers of two or more monomers chosen from acrylic acid, methacrylic acid, acrylic esters and methacrylic esters; copolymers of vinylpyrrolidone and ammonium acryloyldimethyl taurate; copolymers of ammonium acryloyldimethyl taurate and monomers chosen from esters of methacrylic acid and ethoxylated fatty alcohols; hydroxyethylcellulose; hydroxypropylcellulose; hydroxypropylguar; glyceryl polyacrylate; glyceryl polymethacrylate; copolymers of at least one C2-, C3- or C4-alkylene and styrene; polyurethanes; hydroxypropyl starch phosphate; polyacrylamide; copolymer of maleic anhydride and methyl vinyl ether crosslinked with decadiene; carob seed flour; guar gum; xanthan; dehydroxanthan; carrageenan; karaya gum; hydrolyzed corn starch; copolymers of polyethylene oxide, fatty alcohols and saturated methylenediphenyl diisocyanate (e.g. PEG-150/stearyl alcohol/SMDI copolymer).

In one embodiment, the composition according to the invention is in the form of a foamable product (mousse) in combination with a device for foaming. It comprises at least one customary foam-imparting substance known for this purpose, e.g. at least one foam-forming surfactant or at least one foam-forming polymer. Devices for foaming are understood as meaning those devices which enable the foaming of a liquid with or without use of a propellant. A suitable mechanical foaming device which can be, used is, for example, a standard commercial pump foamer or an aerosol foaming head. The product is present either in combination with a mechanical pump foaming device (pump foam) or in combination with at least one propellant (aerosol foam) in an amount of from preferably 1 to 20% by weight, in particular from 2 to 10% by weight. Propellants are chosen, for example, from propane, butane, dimethyl ether and fluorinated hydrocarbons. The composition is foamed directly prior to application and incorporated into the hair as foam and can then be rinsed out or left in the hair without rinsing.

In one embodiment, the composition according to the invention is present in the form of a spray product (hair spray), in combination with a spray device. It may be a pump spray, where the spray device is a mechanical pump spray device. It may also be an aerosol spray, where the composition is in combination with a pressure-resistant packaging, a spray head and at least one propellant chosen from propane, butane, dimethyl ether and fluorinated hydrocarbons. An aerosol spray additionally comprises preferably 15 to 85% by weight, particularly preferably 25 to 75% by weight, of a propellant and is bottled in a pressurized container. Suitable propellants are, for example, lower alkanes, such as, for example, n-butane, isobutane and propane, or mixtures thereof, and dimethyl ether or fluorinated hydrocarbons, such as F 152a (1,1-difluoroethane) or F 134 (tetrafluoroethane), and also propellants which are in gaseous form at the pressures under consideration, such as, for example, N₂, N₂O and CO₂, and mixtures of the abovementioned propellants.

A nonaerosol hair spray is sprayed with the help of a suitable mechanically operated spraying device. Mechanical spraying devices are understood as meaning those devices which permit the spraying of a composition without use of a propellant. A suitable mechanical spray device used may, for example, be a spray pump or an elastic container provided with a spray valve in which the cosmetic composition according to the invention is bottled-under pressure, where the elastic container expands and from which the agent is continuously dispensed as a result of the elastic container contracting upon opening the spray valve.

The composition according to the invention can also be present in the form of a shampoo in combination with at least one cleaning-active surfactant, preferably at least one anionic, amphoteric or nonionic surfactant. The cleaning-active surfactant is present in an amount of from preferably 1 to 50% by weight, preferably from 3 to 30% by weight, in particular from 5 to 20% by weight.

Suitable anionic surfactants are, for example, salts and esters of carboxylic acids, alkyl ether sulfates and alkyl sulfates, fatty alcohol ether sulfates, sulfonic acid and its salts (e.g. sulfosuccinates or fatty acid isethienates), phosphoric esters and their salts, acylamino acids and their salts. A detailed description of these anionic surfactants is to be found in the publication “FIEDLER—Lexikon der Hilfsstoffe [Lexikon of auxiliaries]”, Volume 1, 5th Edition (2002), pages 97 to 102, to which reference is hereby expressly made. Suitable anionic surfactants are, for example, chosen from the alkali metal or alkaline earth metal salts of the C10- to C18-alkyl sulfates, the C10- to C18-alkylsulfonates, the C10- to C18-alkylbenzene-sulphonates, the C10- to C18-xylenesulfonates and the C10- to C18-alkyl ether sulfates ethoxylated with 1 to 10 ethylene oxide units, the ethoxylated sulfosuccinic half-esters of the formula

R¹(OCH₂CH₂)ₘ—O₂C—CH₂CH(SO₃M)-CO₂M,

where R1 is a C10- to C18-alkyl radical, M is an alkali metal or alkaline earth metal cation and m is an integer from 1 to 10; the alkyl ether carboxylates of the formula R²(OCH₂CH₂)ₙ—OCH₂COOM, where R2 is a C10- to C18-alkyl radical, M is an alkali metal or alkaline earth metal cation and n is an integer from 1 to 20. Preferred surfactants are alkyl ether sulfates, alkyl sulfates and alkylsulfonates, where the alkali metal and alkaline earth metal salts of the C10- to C18-alkyl ether sulfates ethoxylated with 1 to 10 ethylene oxide units, in particular sodium lauryl ether sulfate, are particularly preferred. Of the alkyl sulfates, sodium lauryl sulfate is preferred. Of the suitable alkylsulfonates, the sodium salts of the secondary C12- to C16-alkanesulfonates, and mixtures thereof, are preferred. Of the suitable alkylbenzenesulfonates, the sodium salt of the linear dodecylbenzenesulfonate is preferred. Of the suitable alkyl ether sulfates, preference is given to one which has a C12- to C16-alkyl, preferably a lauryl, radical and is ethoxylated with 2 to 4, preferably 3, ethylene oxide units (INCI: Sodium Laurethsulfate). Of the suitable ethoxylated sulfosuccinic half-esters, preference is given to the one which is ethoxylated with 2 to 4, preferably 3, ethylene oxide units and has a C12- to C16-alkyl radical, preferably a lauryl radical. Of the alkyl ether carboxylates, preference is given to one which is ethoxylated with 8 to 14, preferably 10, ethylene oxide units and has a C12- to C16-alkyl radical, preferably a lauryl radical.

Suitable nonionic surfactants are, for example,
ethoxylated fatty alcohols, fatty acids, fatty acid glycerides or alkylphenols, in particular addition products of from 2 to 30 mol of ethylene oxide and/or 1 to 5 mol of propylene oxide onto C8- to C22- or C12- to C18-fatty alcohols, onto C12- to C22-fatty acids or onto alkyl phenols having 8 to 15 carbon atoms in the alkyl group,.
C12- to C22-fatty acid mono- and diesters of addition products of, from 1 to 30 mol of ethylene oxide onto glycerol,
addition products of from 5 to 60 mol of ethylene oxide onto castor oil or onto hydrogenated castor oil,
fatty acid sugar esters, in particular esters of sucrose and one or two C8- to C22-fatty acids, INCI: Sucrose Cocoate, Sucrose Dilaurate, Sucrose Distearate, Sucrose Laurate, Sucrose Myristate, Sucrose Oleate, Sucrose Palmitate, Sucrose Ricinoleate, Sucrose Stearate,
esters of sorbitan and one, two or three C8- to C22-fatty acids and; a degree of ethoxylation of from 4 to 20,
polyglyceryl fatty acid esters, in particular of one, two or more C8- to C22-fatty acids and polyglycerol having preferably 2 to 20 glyceryl units,
fatty acid alkanolamides
alkyl glucosides, alkyl oligoglucosides and alkyl polyglucosides having C8 to C22-alkyl groups, e.g. decylglucoside or laurylglucoside.

Suitable amphoteric surfactants are, for example, derivatives of aliphatic quaternary ammonium, phosphonium and sulfonium compounds of the formula

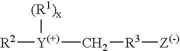

where R2 is a straight-chain or branched-chain alkyl, alkenyl or hydroxyalkyl group having 8 to 18 carbon atoms and 0 to about 10 ethylene oxide units and 0 to 1 glycerol unit; Y is an N, P or S atom; R1 is an alkyl or monohydroxyalkyl group having 1 to 3 carbon atoms; X is 1 if Y is a sulfur atom, and X is 2 if Y is a nitrogen atom or a phosphorus atom; R3 is an alkylene or hydroxyalkylene group having 1 to 4 carbon atoms and Z⁽⁻⁾ is a carboxylate, sulfate, phosphonate or phosphate group.

Other amphoteric surfactants, in particular betaines, are likewise suitable for the hair-cleansing compositions according to the invention. Examples of betaines include C8- to C18-alkylbetaines, such as cocodimethylcarboxymethylbetaine, lauryldimethylcarboxymethylbetaine, lauryldimethylalphacarboxyethylbetaine, cetyldimethylcarboxymethylbetaine, oleyldimethylgammacarbbxypropylbetaine and laurylbis(2-hydroxypropyl)alphacarboxyethylbetaine; C8- to C18-sulfobetaines, such as cocodimethylsulfopropylbetaine, stearyldimethylsulfopropylbetaine, lauryldimethylsulfoethylbetaine, laurylbis(2-hydroxyethyl)sulfopropylbetaine; the carboxyl derivatives of imidazole, the C8- to C18-alkyldimethylammonium acetates, the C8- to C18-alkyldimethylcarbonylmethylammonium salts, and the C8- to C18-fatty acid alkylamidobetaines such as, for example, coconut fatty acid amidopropylbetaine (INCI: Cocamidopropylbetaine) and N-coconut fatty acid amidoethyl-N-[2-(carboxymethoxy)ethyl]glycerol (INCI: Cocoampho carboxyglycinate). Particular preference is given to a surfactant mixture of lauryl ether sulfate and cocamidopropylbetaine.

In a further embodiment, the fluorescent perylene derivatives can also be used directly as crystalline solid, e.g. as powder or granules. This solid can be mixed directly prior to use with one of the abovementioned application forms (in particular lotions, fluid gels etc.) and applied to the human hair. The preferably pulverulent or granular solid can be used here in up to 100% pure form or it can be mixed with inert solids, which are likewise preferably pulverulent or granular, as support material. The solid can also be coated with nonvolatile, wetting liquids, in particular oils, to avoid or suppress dust formation during use. Suitable oils are, in particular, hydrophobic oils liquid at room temperature (25° C.), e.g. vegetable or animal oils, mineral oils (Paraffinum liquidum), silicone oils or mixtures thereof. Hydrocarbon oils, e.g. paraffin or isoparaffin oils, squalane, oils from fatty acids and polyols, in particular triglycerides, are suitable. Suitable vegetable oils are, for example, sunflower oil, coconut oil, castor oil, lanolin oil, jojoba oil, corn oil, soybean oil etc.

The examples below are intended to illustrate the subject matter of the invention in more detail.

### EXAMPLES

Perylene compounds to be used in the examples:
1) N,N′-bis(1-butylpentyl)perylene-3,4:9,10-bis(dicarboximide)
2) N,N′-bis(1-hexylheptyl)perylene-3,4:9,10-bis(dicarboximide)
3) N,N′-bis(1-heptyloctyl)perylene-3,4:9,10-bis(dicarboximide)
4) N,N′-bis(1-octylnonyl)perylene-3,4:9,10-bis(dicarboximide)
5) N,N′-bis(2,5-di-tert-butylphenyl)perylene-3,4:9,10-bis(dicarboximide)
6) N-(1-hexylheptyl)perylene-3,4-dicarboximide
7) N-(2,5-di-tert-butylphenyl)perylene-3,4-dicarboximide
8) N,N′-bis(l-hexylheptyl)benzo[ghi]perylene-2,3,8,9,11,12-hexacarboxylic 2,3;8,9-bis(dicarboximide) 11,12-anhydride
9) N,N′-bis(1-hexylheptyl)benzo[ghi]perylene-2,3,8,9,11,12-hexacarboxylic tris(dicarboximide)
10) N,N′-bis(2,5-di-tert-butylphenyl)thieno-[2′,3′,4═,5′:4,5]phenanthro[2,1,10-def:7,8,9-d′e′f′]diisoquinolino-2H,7H-1,3,6,8-tetrone
11) N,N′-bis(hexylheptyl)thieno[2′,3′,4′,5′:4,5]-phenanthro[2,1,10-def:7,8,9d′e′f′]diisoquinolino-2H,7H-1,3,6,8-tetrone
12) 1-[N^{1′}(N^{2′}-(1-hexylheptyl)perylene-3,4:9,10-bis(dicarboximide)]-N¹,N²-bis(1-hexylheptyl)perylene-3,4:9,10-bis(dicarboximide)

### Synthesis Example

### Synthesis of 1-[N^{1′}-(N^{2′}-(1-hexylheptyl)perylene-3,4:9,10-bis(dicarboximide)]-N′,N²-bis(1-hexylheptyl)perylene-3,4:9,10-bis(dicarboximide)

2.17 g (2.83 mmol) of 1-amino-N,N′-bis(1-hexylheptyl)perylene-3,4:9,10-bis(dicarboximide)¹⁾, 1.77 g (3.10 mmol) of N-(1-hexylheptyl)perylene-3,4:9,10-tetracarboxylic 3,4-anhydride 9,10-carboximide²⁾, 0.128 g (0.82 mmol) of zinc acetate dihydrate and 15 g of imidazole are stirred at 150° C. for 45 minutes. After cooling, the mixture is rinsed from the reaction vessel with 50 ml of ethanol, poured into 250 ml of 2 N hydrochloric acid and stirred for a further hour at room temperature. The precipitated dye is filtered off with suction over a glass frit and dried under reduced pressure at 40° C. Purification is carried out using column chromatography over silica gel with toluene as eluent. The product is eluted here as a pale red luminous band as concentrated fraction. Impurities, primarily the N-(1-hexylheptyl)perylene-3,4:9,10-tetracarboxylic 3,4-anhydride 9,10-carboximide used in excess, have lower R_{f} values and can thus be separated off easily from the main product. The product fraction, which appears thin-layer chromatographically uniform, is concentrated on a rotary evaporator, after cooling slowly precipitated out with cold methanol, filtered with suction and dried.

Yield: 2.40 g (64%) red powder with m.p.>300° C.

UV/vis (chloroform):ₘₐₓ=sh 466.4 nm, 492.9, sh 525.0, 529.7

1) H. Langhals, S. Kirner, Eur. J. Org. Chem. 2000, 365-380.

2) H. Kaiser, J. Lindner, H. Langhals, Chem. Ber. 1991, 124, 529-535.

Example 1: Hair rinse

| | | |
|---|---|---|
| 4.3 | g | cetearyl alcohol (Lanette ® O) |
| 0.4 | g | cetyl lactate |
| 0.5 | g | vaseline |
| 1.2 | g | cetyltrimethylammonium chloride |
| 0.45 | g | polyvinylpyrrolidone |
| 0.3 | g | perylene compound 1, 2, 3, 4, 5, 6, 7, 8, 9, |
| | | 10, 11 and/or 12 |
| ad 100 | g | water |

Example 2: Hair treatment

| | | |
|---|---|---|
| 5.5 | g | cetearyl alcohol (Lanette ® O) |
| 1.2 | g | vaseline |
| 1.0 | g | Paraffinum liquidum |
| 0.5 | g | dimethylpolysiloxane (Belsil ® DM 500) |
| 0.3 | g | lanolin alcohol |
| 0.2 | g | lanolin |
| 1.2 | g | cetyltrimethylammonium chloride |
| 0.3 | g | citric acid |
| 0.4 | g | perfume |
| 0.3 | g | perylene compound 1, 2, 3, 4, 5, 6, 7, 8, 9, |
| | | 10, 11 and/or 12 |
| ad 100 | g | water |

Example 3: Cream-like hair treatment

| | | |
|---|---|---|
| 6 | g | cetearyl alcohol |
| 1.7 | g | glycerol |
| 1 | g | cetyltrimethylammonium chloride |
| 1 | g | vegetable oil |
| 0.5 | g | panthenol |
| 0.5 | g | silicone oil (dimethylpolysiloxane) |
| 0.2 | g | perfume |
| 0.3 | g | perylene compound 1, 2, 3, 4, 5, 6, 7, 8, 9, |
| | | 10, 11 and/or 12 |
| ad 100 | g | water |

Example 4: Leave-on spray hair treatment

| | | |
|---|---|---|
| 0.5 | g | cetyl alcohol |
| 0.3 | g | glycerol |
| 0.25 | g | cetyltrimethylammonium chloride |
| 0.2 | g | styrene/vinylpyrrolidone copolymer |
| 0.2 | g | panthenol |
| 0.3 | g | perylene compound 1, 2, 3, 4, 5, 6, 7, 8, 9, |
| | | 10, 11 and/or 12 |
| 0.2 | g | perfume |
| 4.8 | g | ethanol |
| ad 100 | g | water |

Example 5: Hair wax

| | | |
|---|---|---|
| 10 | g | triceteareth-4 phosphate |
| 6 | g | PEG-200 Hydrogenated Glyceryl Palmate |
| 18 | g | caprylic/capric triglyceride |
| 0.5 | g | perfume |
| 0.5 | g | preservative |
| 0.1 | g | perylene compound 1, 2, 3, 4, 5, 6, 7, 8, 9, |
| | | 10, 11 and/or 12 |
| ad 100 | g | water |

Example 6: Hair shampoo

| | | |
|---|---|---|
| 30 | g | sodium lauryl ether sulfate |
| 8 | g | cocamidopropylbetaine |
| 3 | g | glycol distearate |
| 0.5 | g | perfume |
| 0.35 | g | sodium benzoate |
| 0.15 | g | sodium formate |
| 0.2 | g | sodium chloride |
| 0.1 | g | perylene compound 1, 2, 3, 4, 5, 6, 7, 8, 9, |
| | | 10, 11 and/or 12 |
| ad 100 | g | water |

Example 7: Cream shower bath

| | | |
|---|---|---|
| 12 | g | 70% strength sodium lauryl ether sulfate |
| 0.7 | g | preservative |
| 0.4 | g | perfume |
| 7.5 | g | opacifier (20% by weight PEG-3 Distearate and |
| | | 18% by weight sodium lauryl ether sulfate in |
| | | water) |
| 0.3 | g | Polyquaternium-10 (cationic cellulose) |
| 0.4 | g | PEG-7 Glyceryl Cocoate |
| 0.3 | g | perylene compound 1, 2, 3, 4, 5, 6, 7, 8, 9, |
| | | 10, 11 and/or 12 |
| 0.3 | g | D-panthenol |
| 5 | g | cocamidopropylbetaine, 30% strength in water |
| 1 | g | NaCl |
| ad 100 | g | water |

1. The use of fluorescent perylene compounds for the cosmetic treatment of human hair.

2. The use as claimed in claim 1 for improving the shine or the luminosity of human hair.

3. The use as claimed in claim 1, characterized in that the perylene compound is substituted in at least one of the positions 3, 4, 9 or 10 by a carbonyl group.

4. The use as claimed in claim 1, characterized in that the perylene compound is substituted by a group bridging positions 3, 4 and/or positions 9, 10, where the bridging group is chosen from —CO—NR1-CO—; 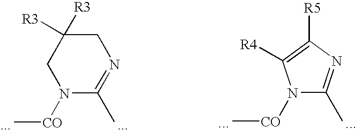
where R1 is linear or branched C1-C24-alkyl groups or is chosen from 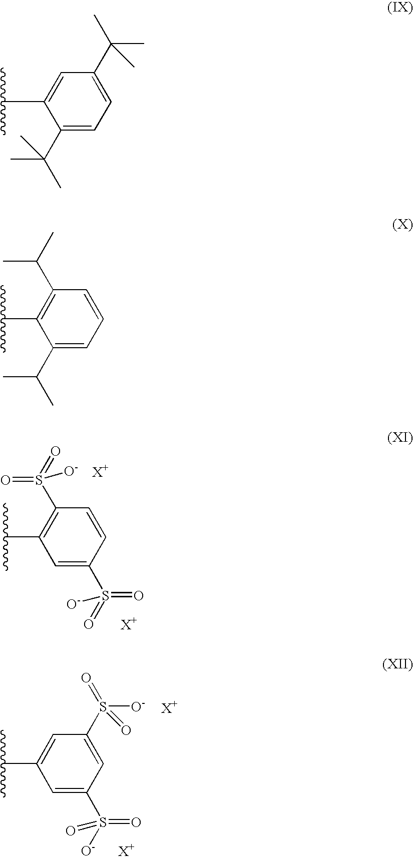
where X⁺ is a proton or a cation; R3 is a linear C1-C9-alkyl group; R4 and R5 together form a fused-on hydrocarbon radical.

5. The use as claimed in claim 1, characterized in that the perylene compound is chosen from 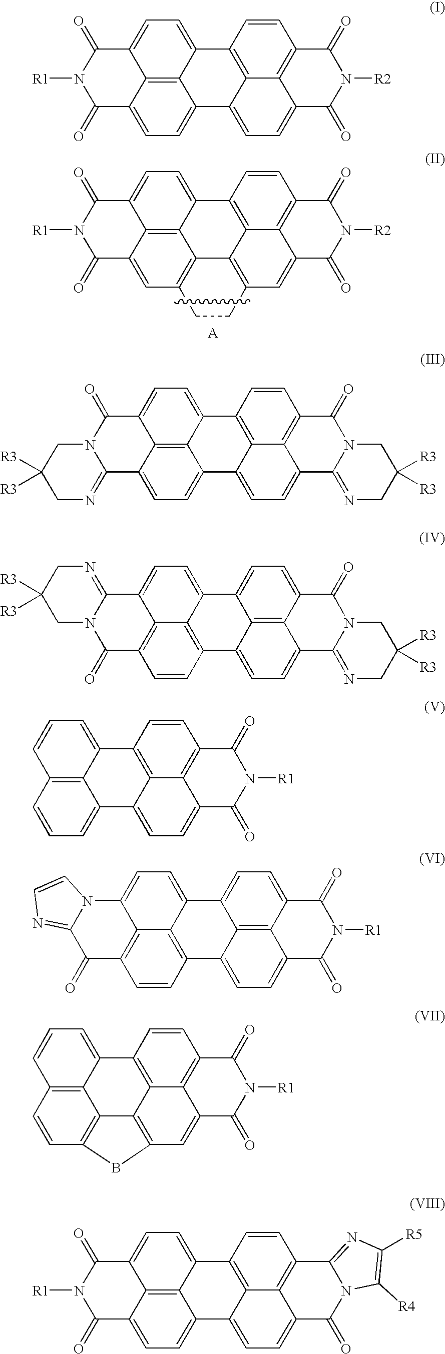 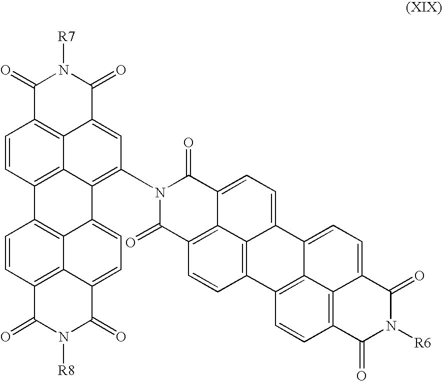
in which R1 and R2, independently of one another, are linear or branched C₁-C₂₄-alkyl groups or are chosen from radicals of the general formulae (IX) to (XII) as in claim 4, and R3, R4 and R5 have the same meaning as in claim 4 and in which R6, R7 and R8, independently of one another, are linear or branched C₁-C₂₄-alkyl groups, and A and B are divalent connecting groups.

6. The use as claimed in claim 1, characterized in that the perylene compound is chosen from N,N′-bis(alkyl)perylene-3,4:9,10-bis(dicarboximide, N-alkylperylene-3,4-dicarboximide, N,N′-bis(alkyl)benzo[ghi]perylene-2,3,8,9,11,12-hexacarboxylic 2,3;8,9-bis(dicarboximide) 11,12-anhydride, N,N′-bis(alkyl)benzo[ghi]perylene-2,3,8,9,11,12-hexacarboxylic tris(dicarboximide), N,N′-bis(alkyl)thieno-[2′,3′,4′,5′:4,5]phenanthro[2,1,10-def:7,8,9-d′e′f′]-diisoquinolino-2H,7H-1,3,6,8-tetrone, 1-[N^{1′}-(N^{2′}-(alkyl)perylene-3,4:9,10-bis(dicarboximide)]-N¹,N²-bis(alkyl)perylene-3,4:9,10-bis(dicarboximide).

7. The use of a fluorescent perylene compound as claimed in claim 1 for producing a composition for the treatment of human hair, where the composition is chosen from hair waxes, hair treatments, hair lotions, hair gels, hair creams, hair foams, hair sprays and shampoos.

8. A hair-treatment composition which is suitable for the treatment of human hair, comprises at least one fluorescent perylene compound and is either
present as a hair wax with a content of at least one wax substance or
is present as a hair treatment or hair lotion in the form of a preparation comprising at least one additional haircare substance or
is present as a hair gel with at least one gel former or
is present as an aerosol or pump foam in combination with a device for foaming or
is present as a hair spray in combination with a spray device or
is present as a shampoo with a content of at least one cleaning-active surfactant.

9. A perylene compound of the general formula 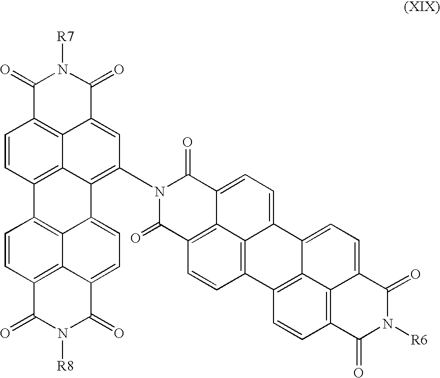
in which R6, R7 and R8, independently of one another, are linear or branched C₁-C₂₄-alkyl groups.

10. 1-[N^{1′}-(N^{2′}-(alkyl)perylene-3,4:9,10-bis(dicarboximide)]-N¹,N²-bis(alkyl)perylene-3,4:9,10-bis(dicarboximide).
